(19) United States
(12) Patent Application Publication
(10) Pub. No.: US 2021/0321888 A1
(43) Pub. Date: Oct. 21, 2021

(54) VOLUMETRIC BLOOD FLOW

(71) Applicant: THE UNIVERSITY OF SHEFFIELD, Sheffield (GB)

(72) Inventors: Paul Morris, Sheffield (GB); Julian Gunn, Sheffield (GB); David Rodney Hose, Sheffield (GB); Patricia Lawford, Sheffield (GB)

(21) Appl. No.: 17/267,751

(22) PCT Filed: Aug. 8, 2019
(86) PCT No.: PCT/GB2019/052228

§ 371 Date: Feb. 10, 2021

(30) Foreign Application Priority Data
Aug. 13, 2018 (GB) 1813170.6

(51) Int. Cl. A61B 5/0215; A61B 5/026; A61B 34/10

(52) U.S. Cl. CPC A61B 5/02158; A61B 34/10; A61B 5/026

(57) ABSTRACT

A computer-implemented method of determining data indicating volumetric flow rate within an artery. The method comprises receiving data indicating a proximal pressure measurement relative to a direction of blood flow; receiving data indicating a distal pressure measurement relative to the direction of blood flow; wherein the proximal pressure measurement and the distal pressure measurement are obtained using at least one pressure transducer; receiving geometric model data representing the artery, wherein the geometric model data is generated based upon image data indicative of the artery; and computing a volumetric flow rate with in the artery, wherein computing comprises performing a numerical simulation of blood flow through the artery based upon the geometric model data, the data indicating a proximal pressure measurement and the data indicating a distal pressure measurement.

### TECHNICAL FIELD

The present specification relates to systems and methods for determining data indicating a volumetric blood flow within an artery, in particular a coronary artery.

### BACKGROUND

Arterial disease is a common cause of death and morbidity. Arterial disease is caused by narrowings (stenoses) in the arteries which reduces blood flow. Arterial disease may be treated by medical therapy and with interventional techniques such as surgery or angioplasty. The decision to proceed with intervention is currently based upon physiological assessment.

One common type of arterial disease is coronary artery disease. For coronary artery disease, the current most popular interventional physiological assessment is fractional flow reserve (FFR). FFR is calculated as a ratio between mean distal coronary pressure and mean aortic pressure during maximal hyperaemia (blood flow). FFR is a surrogate marker for maximal flow in an artery in the presence of a stenosis compared to maximal flow in the hypothetical absence of the stenosis. FFR requires hyperaemia to be induced in a patient and an invasive procedure to be performed in which pressure measurements are taken using a pressure wire inserted through a catheter in the patient's artery at the same time as coronary angiography to image the diseased artery.

Recent efforts have focused on estimating FFR, or equivalent measures, using computational fluid dynamics simulations based upon 3D models of a patient's artery generated from arterial imaging. A key motivation for developing such computational fluid dynamics simulations is to minimize the requirement for invasive procedures.

Attempts have also been made to determine an indication of the absolute volumetric flow rate within the coronary artery and the absolute reduction in flow caused by the stenosis. Some methods rely upon surrogate measures such as Doppler ultrasound fluid velocity or thermodilution mean transit time. Another method using a thermodilution technique requires a specific continuous infusion catheter and an hyperaemia-inducing infusion. All of these techniques require additional specialised apparatus and are either inaccurate, time-consuming or technically challenging to perform. What is required is an efficient and accurate method of determining volumetric flow rate through an artery such as a coronary artery and other types of artery.

### SUMMARY

According to a first aspect, there is provided a method for determining data indicating a volumetric flow rate within an artery. The method comprises receiving data indicating a proximal pressure measurement relative to a direction of blood flow; receiving data indicating a distal pressure measurement relative to the direction of blood flow; wherein the proximal pressure measurement and the distal pressure measurement are obtained using at least one pressure transducer; receiving geometric model data representing the artery, wherein the geometric model data is generated based upon image data indicative of the artery; and computing a volumetric flow rate within the artery, wherein computing comprises performing a numerical simulation of blood flow through the artery based upon the geometric model data, the data indicating a proximal pressure measurement and the data indicating a distal pressure measurement.

The proximal pressure measurement is a direct pressure measurement taken proximal to a lesion present in the artery, relative to the direction of blood flow. For example, in the case of a coronary artery, the proximal pressure measurement may be taken in the aorta or arterial ostium. The distal pressure measurement is a direct pressure measurement taken distal to the lesion, relative to the direction of blood flow. Advantageously, the proximal pressure measurement and the distal pressure measurement may be taken using standard apparatus and in the case of a coronary artery, the apparatus used in FFR assessment. For example, a catheter may be used to obtain the proximal pressure measurement and a catheter or pressure wire may be used to obtain the distal pressure measurement. In addition, the pressure measurements may be taken under resting conditions and the method does not require pressure measurements to be taken under the conditions of hyperaemia if resting flow rate is to be computed. Alternatively, the method is also applicable under conditions of hyperaemia if hyperaemic flow rate is to be computed.

Realised by the inventors is the ability to accurately and efficiently determine volumetric flow rate by performing a numerical simulation of blood flow in a patient's artery based upon pressure measurements taken directly from the patient's artery and geometric model data representative of the patient's artery. By performing a numerical simulation based upon pressure measurements taken directly from the patient's artery and geometric model data derived from medical imaging of the patient's artery, the numerical simulation can be made patient-specific and the volumetric flow rate may be accurately computed for the patient.

The volumetric flow rate is a physiological parameter measured in units of volume per unit time, for example, millilitres per second (ml/s). The determined data indicating a volumetric flow rate provides data indicating the flow rate through an individual artery. By contrast, some cardiac MRI-based prior art methods provide only an indication of flow supplying a territory of myocardium and cannot provide the flow rate through an individual (coronary) artery. Some thermo-dilution based prior art methods attempt to estimate volumetric flow rate but require passage of a specific continuous infusion catheter and a hyperaemia inducing infusion. Such a method requires additional specialised apparatus, is less accurate in estimating volumetric flow and involves a procedure with greater risk.

FFR is a pressure derived estimate of flow reduction and provides an estimate of the fractional reduction in flow as compared to a hypothetical ideal. In addition, as FFR is a ratio of pressures only, any abnormal flow rates may be hidden by similar pressure values. Furthermore, FFR provides limited physiological information and provides no information for differentiating between epicardial and microvascular disease, of which the microvasculature may be a key influence on FFR and other pathological states. By comparison, the volumetric flow rate (in combination with the pressure measurements) may be used to differentiate between epicardial and microvascular disease and provides an improved indication of the health of the entire coronary arterial system as compared to FFR.

The numerical simulation may be a computational fluid dynamics (CFD) simulation.

The method may further comprise setting one or more boundary conditions of the numerical simulation based upon the data indicating a proximal pressure measurement and the data indicating a distal pressure measurement. Setting one or more boundary conditions may comprise: setting an inlet pressure boundary condition based upon the data indicating a proximal pressure measurement; and setting an outlet pressure boundary condition based upon the data indicating a distal pressure measurement.

The method may further comprise determining one or more physiological parameters associated with the artery based upon the computed volumetric flow rate. The one or more physiological parameters may comprise one or more of the following: stenosis resistance, distal microvascular resistance, coronary microvascular resistance and coronary flow reserve. It will be appreciated that the method is capable of determining a stenosis resistance and distal microvascular resistance under either baseline or maximal hyperaemic flow conditions. In the case of the coronary circulation, this is analogous to baseline and hyperaemic stenosis resistance (BSR, HSR) and microvascular resistance (BMR and HMR). Given that volumetric flow rate can be accurately computed, additional physiological parameters can also be accurately computed such that improved information associated with the health of a patient's artery is provided.

The method may further comprise receiving image data representing the artery; generating a three dimensional model representing the artery based upon the image data; discretizing the three dimensional model; and generating the geometric model data representing the artery based upon the discretized three dimensional model.

The artery may be a coronary artery. The received geometric model data may be generated based upon image data representing the coronary artery. The image data may be based upon a coronary angiogram. Angiography provides detailed images of a patient's artery in high enough resolution for accurate construction of a model of the patient's artery.

The at least one pressure transducer may comprise a catheter and/or pressure wire.

According to a second aspect there is provided a computer apparatus for determining data indicating volumetric flow rate within an artery comprising: a memory storing processor readable instructions; and a processor arranged to read and execute instructions stored in said memory; wherein said processor readable instructions comprise instructions arranged to control the computer to carry out a method according to the first aspect.

According to a third aspect, there is provided a computer readable medium carrying computer readable instructions configured to cause a computer to carry out a method according to the first aspect.

According to a fourth aspect, there is provided an apparatus for determining data indicating volumetric flow rate within an artery comprising: a computer readable medium carrying computer readable instructions configured to cause a computer to carry out a method according to the first aspect; and a pressure transducer for obtaining a pressure measurement in an artery.

Aspects can be combined and it will be readily appreciated that features described in the context of one aspect can be combined with other aspects.

It will be appreciated that aspects can be implemented in any convenient form. For example, aspects may be implemented by appropriate computer programs which may be carried on appropriate carrier media which may be tangible carrier media (e.g. disks) or intangible carrier media (e.g. communications signals). Aspects may also be implemented using suitable apparatus which may take the form of programmable computers running computer programs.

### BRIEF DESCRIPTION OF THE DRAWINGS

Embodiments will now be described, by way of example, with reference to the accompanying drawings, in which:

FIG. 1 is a schematic illustration of a system according to an embodiment.

FIG. 1A is a schematic illustration of a computer of the system of FIG. 1 in more detail.

FIG. 2 is a flowchart showing processing carried out for determining data indicating volumetric flow rate within an artery.

FIG. 3 is a schematic illustration of processing carried out for determining volumetric flow rate within a coronary artery.

FIG. 4 is a flowchart showing additional processing that may be carried out for determining data indicating volumetric flow rate within an artery.

FIG. 5 is an exemplary flow circuit test-rig for use in validating the method.

FIGS. 6A and 6B show Bland Altman plots according to an example validation.

FIG. 7 is a table of results associated with an example.

FIG. 8 shows an exemplary diseased left anterior descending artery and computed physiological parameter data.

FIG. 9 shows another exemplary diseased left anterior descending artery and computed physiological parameter data.

FIG. 10 shows an example of computed volumetric flow through a diseased left circumflex artery for both baseline and hyperaemic conditions over two cardiac cycles.

FIG. 11 shows example physiological parameter data that may be computed.

### DETAILED DESCRIPTION

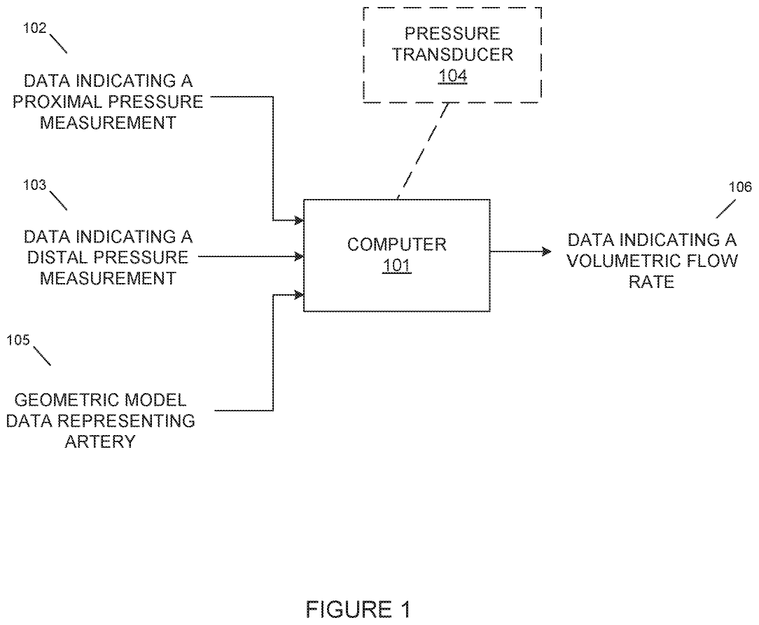

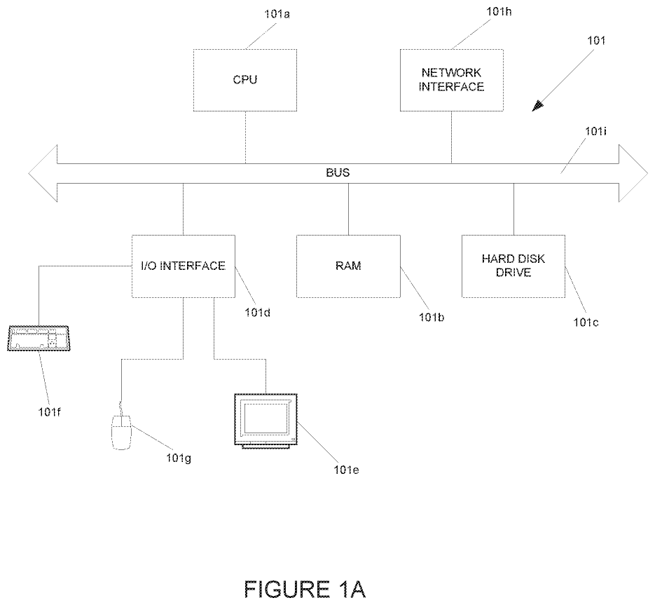

Referring now to FIG. 1, a computer 101 is arranged to receive data indicating a proximal pressure measurement 102 and data indicating a distal pressure measurement 103. The proximal pressure measurement and the distal pressure measurement are obtained using at least one pressure transducer 104. For example, in the case of a coronary artery, the proximal pressure measurement may be obtained using a cardiac catheter and the distal pressure measurement may be obtained using a pressure wire. The at least one pressure transducer 104 is optionally in communication with the computer 101.

The proximal pressure measurement is a direct pressure measurement taken proximal to a lesion present in an artery, relative to the direction of blood flow. The distal pressure measurement is a direct pressure measurement taken distal to the lesion, relative to the direction of blood flow. The proximal pressure measurement and the distal pressure measurement may be a transient (dynamic) dataset or an average pressure over time. The proximal pressure measurement and the distal pressure measurement may be taken using a standard catheter and pressure wire used in the FFR procedure in the case of a coronary artery. For example, Verrata (pressure guide wire) manufactured by Philips Volcano, California, USA is a suitable pressure wire.

Advantageously, the procedure for taking pressure measurements using a pressure transducer such as a catheter and pressure wire is within the standard competency of an interventional cardiologist and the required pressure measurements can be taken without the need for additional wires, catheters, drugs, risk or procedural time as compared to the FFR procedure.

The computer 101 is further arranged to receive geometric model data 105 representing the artery. For example, the geometric model data 105 may be a mesh model representing the geometry of the artery. The geometric model data 105 may be in a format suitable for performing a numerical simulation. For example, the model data may be a volumetric mesh representing the closed surfaces of the artery. The data format of the geometric model data 105 may be dependent on a computer program used to perform the numerical simulation.

The computer 101 is arranged to generate a volumetric flow rate within the artery by performing a numerical simulation of blood flow through the artery based upon the geometric model data 105 representing the artery, the data indicating a proximal pressure measurement 102 and the data indicating a distal pressure measurement 103.

The numerical simulation may be a computational fluid dynamics (CFD) simulation and the volumetric flow rate may be an output of the simulation. The CFD simulation may be performed by a CFD solver such as ANSYS-CFX or ANSYS Fluent produced by Ansys, Inc. Pennsylvania, USA. The simulation may solve the unsteady momentum (Navier-Stokes) and continuity equations, in one, two or three dimensions, with the conservation form of the finite volume technique.

The data indicating a proximal pressure measurement 102 and the data indicating a distal pressure measurement 103 may be used to set one or more boundary conditions of the numerical simulation. For example, an inlet pressure boundary condition may be set based upon the data indicating a proximal pressure measurement 102 and an outlet pressure boundary condition may be set based upon the data indicating a distal pressure measurement 103. Typically prior art CFD simulations of an artery, and in particular, a coronary artery, are performed using estimated boundary conditions. The inventors have realised, however, that it is possible to generate accurate, patient specific boundary conditions for a patient based upon data that may be obtained readily using current procedures, and that use of such boundary conditions in the simulation allows improved information to be generated using existing simulations. In particular, by performing a numerical simulation of blood flow in a patient's artery based upon pressure measurements taken directly from the patient's artery and geometric model data representative of the patient's artery, the volumetric flow rate within the patient's artery can be computed accurately and efficiently for the patient.

The computer 101 is further arranged to output data 106 indicating the computed volumetric flow rate within the artery.

Alternatively, or in combination, the computer 101 may be in communication with a remote sever. The remote server may be configured to perform the numerical simulation. The computer 101 may transmit the data indicating a proximal pressure measure 102, the data indicating a distal pressure measurement 103 and the geometric model data 105 to the remote server to perform the numerical simulation. The remote server may transmit a result of the simulation, such as the data indicating a volumetric flow rate to the computer 101 for output.

FIG. 1A shows the computer 101 of FIG. 1 in further detail. It can be seen that the computer 101 comprises a CPU 101a which is configured to read and execute instructions stored in a volatile memory 101b which takes the form of a random access memory. The volatile memory 101b stores instructions for execution by the CPU 101a and data used by those instructions. For example, in use, the received data indicating a proximal pressure measurement 102, the data indicating a distal pressure measurement 103 and the geometric model data 105 may be stored in volatile memory 101b.

The computer 101 further comprises non-volatile storage in the form of a hard disc drive 101c. The computer 101 further comprises an I/O interface 101d to which are connected peripheral devices used in connection with the computer 101. More particularly, a display 104e is configured so as to display output from the computer 101. The display 104e may, for example, display the output of the numerical simulation at a particular display resolution. Input devices are also connected to the I/O interface 101d. Such input devices include a keyboard 101f and a mouse 101g which allow interaction with the computer 101. Other input devices may also include gesture-based input devices. A network interface 101h allows the computer 101 to be connected to an appropriate computer network so as to receive and transmit data from and to other computing devices. The CPU 101a, volatile memory 101b, hard disc drive 101c, I/O interface 101d, and network interface 101h, are connected together by a bus 101i.

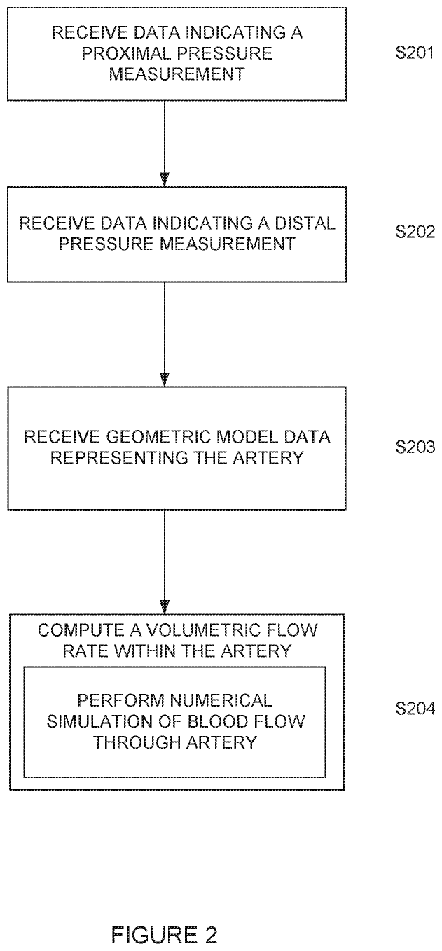

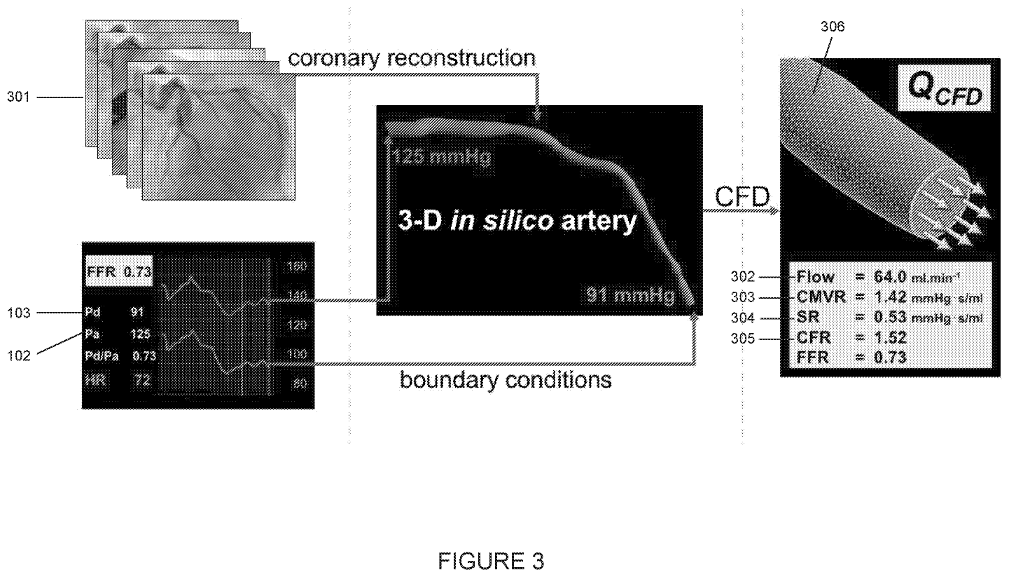

Referring now to FIG. 2, a process for determining data indicating volumetric flow rate within an artery is shown. As will be appreciated, the process may be implemented by the system of FIG. 1, described above. In addition, FIG. 3 shows an exemplary application of the process to a coronary artery.

At step S201, data indicating a proximal pressure measurement 102 relative to a direction of blood flow is received. At step S202, data indicating a distal pressure measurement 103 relative to the direction of blow flood is received. Both the proximal pressure measurements and distal pressure measurements are obtained using at least one pressure transducer 104. For example, as noted above, the proximal pressure measurement may be obtained using a cardiac catheter and the distal pressure measurement may be obtained using a pressure wire in the case of a coronary artery. As noted above, obtaining such pressure measurements is within the standard competence of a cardiologist.

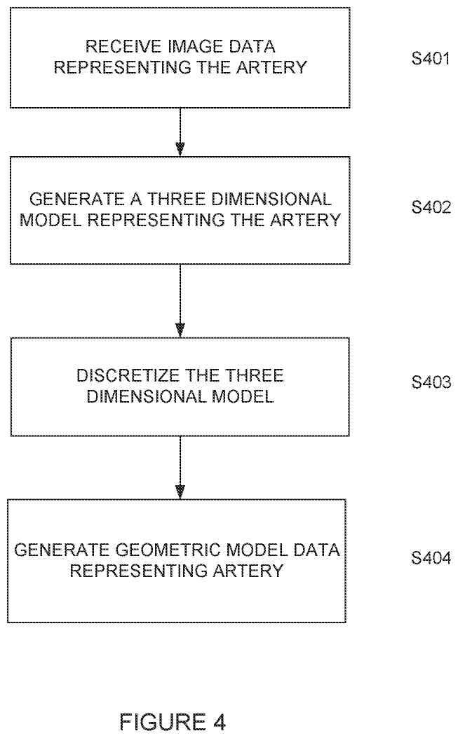

At step S203, geometric model data 105 representing the artery is received. A process for generating such geometric model data 105 is described in more detail below with reference to FIG. 4.

At step S204, the volumetric flow rate within the artery is computed. The computation comprises performing a numerical simulation of blood flow through the artery based upon the geometric model data 105 representing the artery, the data indicating a proximal pressure measurement 102 and the data indicating a distal pressure measurement 103, received in steps S201, S202 and S203.

As noted above, the numerical simulation may be a computational fluid dynamics (CFD) simulation and the volumetric flow rate may be an output of the simulation. The CFD simulation may be performed by a CFD solver such as ANSYS-CFX or ANYSYS Fluent produced by Ansys, Inc. Pennsylvania, USA.

The data indicating a proximal pressure measurement 102 and the data indicating a distal pressure measurement 103 may be used to set one or more boundary conditions of the numerical simulation. For example, an inlet pressure boundary condition may be set based upon the data indicating a proximal pressure measurement 102 and an outlet pressure boundary condition may be set based upon the data indicating a distal pressure measurement 103. This is further illustrated in FIG. 3.

The computed volumetric flow rate 302 may be used to calculate other physiological parameters associated with the artery. In the case of a coronary artery, the physiological parameters may include coronary microvascular resistance 303, epicardial coronary stenosis resistance 304 and coronary flow reserve 305.

For example, coronary microvascular resistance (CMVR) 303 may be computed using the following equation:

$$C M V R = \frac{{P}_{d} - {P}_{v}}{{Q}_{C F D}}$$

where P_{d} is the distal pressure measurement, Pᵥ is a central venous (right atrial) pressure, typically assumed to be zero, and Q_{CFD} is the computed volumetric flow rate 302.

In another example, epicardial stenosis resistance (SR) 304 may be computed as follows:

$$S R = \frac{{P}_{a} - {P}_{d}}{{Q}_{C F D}}$$

where Pₐ is the proximal pressure measurement, P_{d} is the distal pressure measurement and Q_{CFD} is the computed volumetric flow rate 302.

In a further example, coronary flow reserve (CFR) 305 may be computed as follows:

$$C F R = \frac{{Q}_{C F D}^{Hyp}}{{Q}_{C F D}^{B L}}$$

where Q_{CFD}^{Hyp} is the volumetric flow rate computed under conditions of hyperaemia and O_{CFD}^{BL} is the volumetric flow rate computed under baseline or normal resting conditions.

These additional physiological parameters may provide useful information for determining the health of a patient's artery. For example, using the above cardiac-related physiological parameters, it may be possible to differentiate between epicardial and microvascular disease. It may also be useful in determining whether surgical intervention is required. As volumetric flow rate can be accurately computed using the techniques described above, the above physiological parameters can also be accurately computed such that improved information associated with the health of a patient's artery is provided.

Referring now to FIG. 4, a process for generating geometric model data 105 representing an artery is described at a high-level. Further details may be found in Morris P. D. et al. Virtual Fractional Flow Reserve From Coronary Angiography: Modeling the Significance of Coronary Lesions: Results From the VIRTU-1 (VIRTUal Fractional Flow Reserve From Coronary Angiography) Study., JACC Cardiovascular interventions 2013; 6:149-57 which is hereby incorporated by reference in its entirety.

At step S401, image data representing the artery is received. For example, as shown in FIG. 3, the image data 301 may be generated from an angiogram of the patient's artery.

The majority of patients diagnosed with coronary artery disease under consideration for surgical intervention will undergo a coronary angiography to image the patient's coronary artery for inspection. Advantageously, the method can use image data that would be collected under standard procedures. In addition, the required pressure measurements may be taken in the same procedure as a coronary angiogram and therefore, the method does not require a separate procedure for collecting the required data. Angiography provides detailed images of a patient's artery in high enough resolution for accurate construction of a model of the patient's artery. However, it will be appreciated that the process is not limited to image data derived from angiography and other medical imaging images may be suitable as will be apparent to a person skilled in the art.

At step S402, a three dimensional model of the artery is generated based upon the image data. The image data may be segmented to identify the artery and the segmented image data may be used to construct a three dimensional geometric model of the artery. An exemplary three dimensional model is shown in FIG. 3.

Segmentation and three dimensional model generation may be performed using any suitable method. Further details are provided in the above reference.

At step S403, the three dimensional model is discretized. For example, the three dimensional model may be discretized into a volumetric mesh model 306 suitable for performing a numerical simulation as illustrated in FIG. 3.

At step S404, geometric model data 105 is generated based upon the discretized three dimensional model. The format of the geometric model data 105 may be based upon a format required by a numerical simulation program. As described above, the geometric model may be processed in accordance with the processing of FIG. 2 to compute a volumetric flow rate within the artery.

### EXAMPLES

The following examples are provided for illustrative purposes and are not to be construed as being limitations thereon.

### Example 1: Computation of Volumetric Coronary Flow Rate

Volumetric coronary flow rate (Q_{CFD}) was computed from coronary angiographic images (CAG) and pressure data. To compute Q_{CFD}, the three-dimensional (3-D) geometry of the diseased artery was segmented and reconstructed from standard multi-plane CAG. Translesional dynamic pressure data acquired during pressure wire (FFR) assessment were processed. The proximal and distal pressures (Pₐ and P_{d}) were used as boundary conditions for computational fluid dynamics (CFD) simulation. Blood viscosity and density were assumed to be 0.035 Pa·s and 1050 kg/m³ respectively. CFD simulation was performed using ANSYS-CFX (v14.5, PA, USA) on a Dell Precision T5600 computer (Intel Xeon E5 2650, 2 GHz processor, 32 GB RAM). Output data were coronary flow rates in ml/s, referred to as Q_{CFD}.

### Example 2: In Vitro Evaluation

An in vitro experiment for validating the Q_{CFD} computation method was performed using an experimental circuit with specified flow rate. Doppler flow wire analysis was also performed for comparison.

### Experimental Flow Circuit

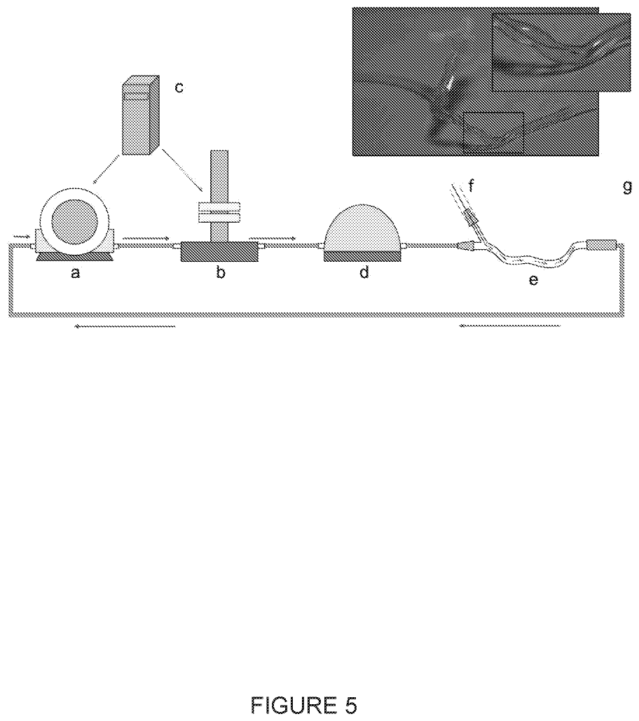

The experimental circuit comprised a steady-flow gear-pump (Pump Head, Cavity Style, TA instruments, MN, USA), compliance chamber, pulsatile manifold (BioDynamic Test Instruments, Bose Corp, ElectroForce Systems Group) (pulsatile experiments only), 3-D printed coronary artery (described below), and fluid reservoir. The system was controlled by WinTest® Control software (version 4.1, Bose Corp, ElectroForce Systems Group). The experimental flow circuit is shown in FIG. 5.

The pump delivered flow rates from 50 to 180 ml/min in 10 ml/min increments, reproducing typical coronary flow rates from baseline through to hyperemic conditions. The bellows displacement system of the pulsatile manifold reproduced a prescribed flow waveform, enabling precise control of the transient waveform and mean flow rate. Pulsatile flow was specified using the patient-specific data derived from invasive clinical measurements. The fluid was a glycerol/water blood analogue with viscosity equivalent to that of blood (0.0035 Pa·s) at room temperature. For a comparative Doppler analysis (described below), an ultrafine nylon powder (Orgasol® Powders, Arkema Group, Paris, FR) was added to mimic the ultrasonic back-scatter properties of erythrocytes. Physiological wires were introduced into the circuit via hemostatic percutaneous coronary intervention (PCI) valves (AccessPLUS Hemostatic Valve, Merit Medical Systems Inc. CA, USA) consistent with standard PCI practice at Sheffield Teaching Hospitals. The system was completely purged of air prior to all analyses.

### Patient-Specific 3-D Arterial Printing

Patients with stable coronary artery disease underwent CAG and pressure-wire assessment (procedure described below). Diseased arteries from five patients were reconstructed in 3-D using the VIRTUheart™ software (The University of Sheffield, Sheffield, UK). These cases represented a range of arteries and stenosis severities (LAD FFR=0.64, LAD FFR=0.72, RCA FFR=0.79, LCX FFR=0.82, RCA FFR=0.86). The 3-D geometry of the arterial lumen was exported and an external wall was defined for 3-D printing (Materialise NV, BE). Connection and access ports were added to the inlets and outlets of each arterial model using computer-aided design without interfering with the reconstructed arterial lumen. The models were printed in TuskXC2700T material by laser stereolithography with a layer thickness of 0.1 mm.

### Experimental Pressure and Flow Measurement

Proximal pressure (Pₐ) was measured in vitro using a TruWave Pressure Transducer (Edwards Lifesciences Corp, CA, US) and distal pressure (P_{d}) with a 0.34 mm Volcano Primewire ([Philips] Volcano, CA, USA), as per standard cardiac catheter laboratory practice. Flow rate and pulsatility were prescribed using the WinTest® Control software. Experimental flow rate (Qₑₓₚ) was calibrated before and after every analysis by measuring the fluid volume draining into a flask in one minute. All experiments were repeated three times (195 separate Q_{CFD} analyses) and mean results calculated for each flow rate.

In Vitro Doppler Analysis

Flow velocity was measured within the experimental circuit with a FloWire® ([Philips] Volcano, CA, USA) which detects flow velocity by pulse wave Doppler (12 MHz) with a range gate of 5-7 mm in a 30° arc. Volcano software was used to track the average peak velocity (APV). The wire was positioned under direct vision and was manipulated until the ‘optimal’ (densest) Doppler signal was observed as per clinical protocol. Volumetric flow rate was calculated directly from Doppler flow velocity and the luminal cross-sectional area (known precisely from the print files) assuming a laminar flow with parabolic profile (APV=2·mean velocity). APV was measured at the arterial inlet and outlet, under both steady-state, and pulsatile flow conditions, at all flow rates, as described above. All measurements were repeated three times at each flow rate and mean results calculated.

### Validation

The flow circuit was run with all five 3-D printed models, at flow rates from 50 to 180 ml/min, in 10 ml/min increments, with steady and pulsatile flows. The pressure gradient was recorded at each flow rate and used to compute Q_{CFD}. The primary outcome measure was the accuracy of Q_{CFD} validated against the gold-standard calibrated experimental flow rate (Qₑₓₚ) over all flow rates. Whereas physiological flows are typically laminar (Re<500), the in vitro protocol had potential to induce supra-physiological flow rates (Re>500) in tighter stenoses at higher flow rates. For this reason, we report separately the accuracy of Q_{CFD} for those cases where Re<500 because this reflects the accuracy of the method over the clinically important, physiological range.

### Statistical Analysis

The accuracy of Q_{CFD} and Q_{Dop} were assessed by comparison with Qₑₓₚ. The mean delta (bias) and the standard deviation of the mean delta (SD) are presented. Bland Altman plots were constructed and the limits of agreement reported (±1.96 SD). Bland Altman limits of agreement comprise 95% of all results. The narrower the Bland Altman limits of agreement, the more accurate a new method is (Q_{CFD}) relative to a gold-standard measure (Qₑₓₚ). Reproducibility was assessed by calculating the coefficient of variation (CoV). Pearson coefficient was used to assess the linear correlation between Qₑₓₚ and both Q_{CFD} and Q_{Dop}. Statistical analysis was performed using SPSS (IBM Corp, NY, US).

### Results: Flow Circuit Hemodynamics and Reproducibility

Across all 3-D printed coronary models, and at all physiological flow rates investigated, the resulting pressure gradients were highly reproducible with a coefficient of variation of 0.04 mmHg. There was no significant difference in the mean (cycle-averaged) pressure gradient whether steady or pulsatile flow was simulated (bias, −0.2 mmHg, SD, 0.9 mmHg).

### Results: Accuracy of Computed Q_{CFD}

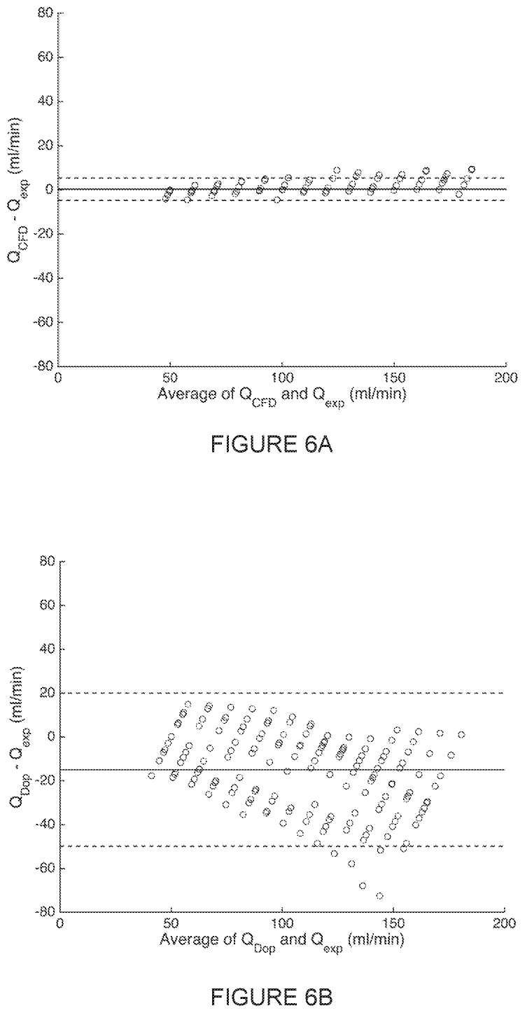

Q_{CFD} demonstrated close agreement with Qₑₓₚ. Across all models, at all flow rates investigated, the mean delta was +2.08 ml/min (SD 3.45) with Bland Altman limits of agreement of −4.7 to +8.8 ml/min (FIG. 6A). Q_{CFD} was closely correlated with Qₑₓₚ (R²=0.999, p<0.001). Q_{CFD} results were reproducible on repeated analysis (CoV=<1 c/o). When supra-physiological cases were excluded from the analysis, accuracy improved slightly and the mean delta was +0.31 ml/min (2.58) with Bland Altman limits of agreement of −4.7 to +5.3 ml/min (FIG. 6A).

### Results: Accuracy of Doppler Flow (Q_{Dop})

Even with direct vision of the wire within the experimental flow circuit, it was difficult to achieve and maintain a consistent Doppler signal, the quality and magnitude of which was sensitive to the slightest movements of the wire. The coefficient of variability for Doppler flow (Q_{Dop}) across all models, at all physiological flow rates was 6.4% when the wire was positioned at the arterial inlet, and 17.4% at the outlet. Just as with Q_{CFD}, Q_{Dop} was independent of whether flow was steady or pulsatile i.e. Average peak (Doppler) Velocity (APV) was dependent upon mean flow rate and was insensitive to flow pulsatility. Despite a strong correlation, Q_{Dop} consistently underestimated Qₑₓₚ (R²>0.98, p<0.001; mean delta −15.0 ml/min). Bland Altman limits of agreement for Q_{Dop} were significantly wider than the limits for Q_{CFD} (−50 to +20 ml/min vs −4.7 to +5.3 ml/min) (FIG. 6B).

### Results: General Conclusions

The computed Q_{CFD} agreed closely with the calibrated experimental flow rate (Qₑₓₚ) with negligible bias and narrow limits of agreement (bias+0.31 ml/min; SD, 2.58, Bland-Altman limits of agreement of −4.7 to +5.3 ml/min, R²=0.999, p<0.001). Q_{CFD} was also reproducible with a coefficient of variability <1%. Doppler ultrasound (Q_{Dop}) results proved to be more variable and inaccurate (bias −15 ml/min, Bland Altman limits of agreement of −50 to +20 ml/min, CoV=6%).

### Example 3: Evaluation in the Catheterization Laboratory and Clinical Validation

Using CAG image data and the measured pressure gradient (as described above), the Q_{CFD} method was evaluated on a cohort of patients (different to Example 2) with stable coronary artery disease undergoing cardiac catheterization and FFR assessment (Volcano Primewire or PressureWire™ X guidewire, St Jude/Abbott). Q_{CFD} was computed (as described above) from reconstructed angiogram images and invasively measured pressures. In addition to computing Q_{CFD}, CMVR, SR and CFR were calculated according to the equations described above.

Each patient also underwent Doppler flow wire assessment (FloWire®, [Philips] Volcano) to derive coronary flow (Q_{Dop}). As discussed previously, there is no gold-standard method for measuring volumetric coronary flow rate in vivo. Therefore, to provide in vivo validation, we “reversed” the computational model and prescribed the flow results (i.e. Q_{Dop} and Q_{CFD}) to the reconstructed arteries in order to simulate the translesional pressure drop. An accurate measurement of flow should reproduce the pressure gradient measured invasively in the catheterization laboratory whereas an inaccurate flow would reproduce a very different gradient/FFR. A second validation was considered by comparing the SR under baseline and hyperemic conditions. Because atherosclerotic plaque acts as a fixed resistance, the SR is expected to remain constant under baseline and hyperemic flow conditions.

### Results: Computed Q_{CFD}

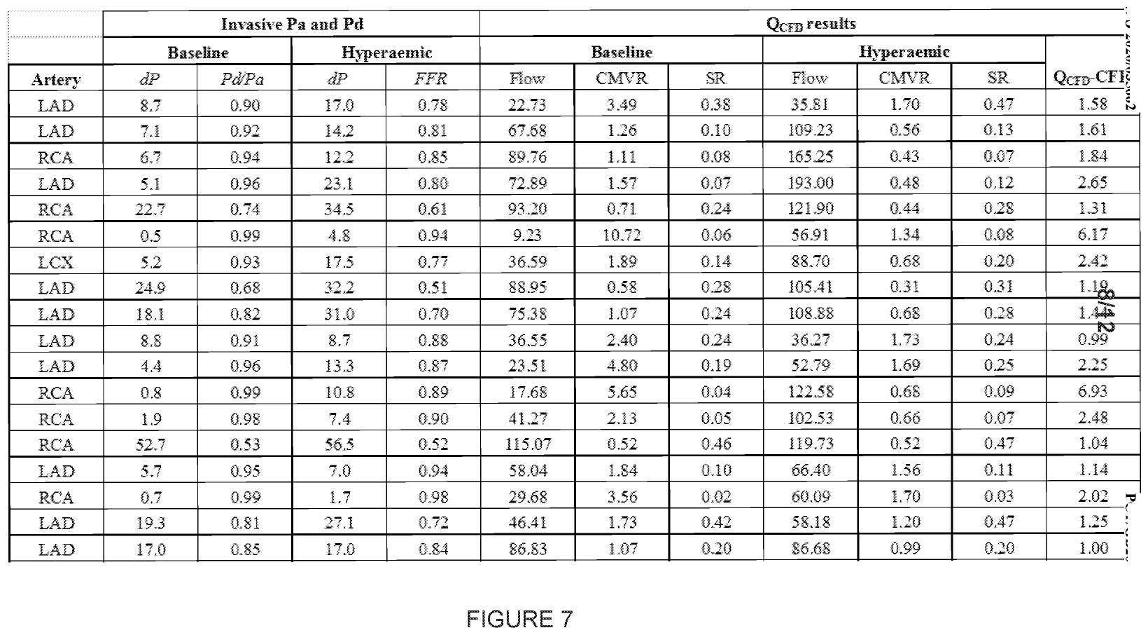

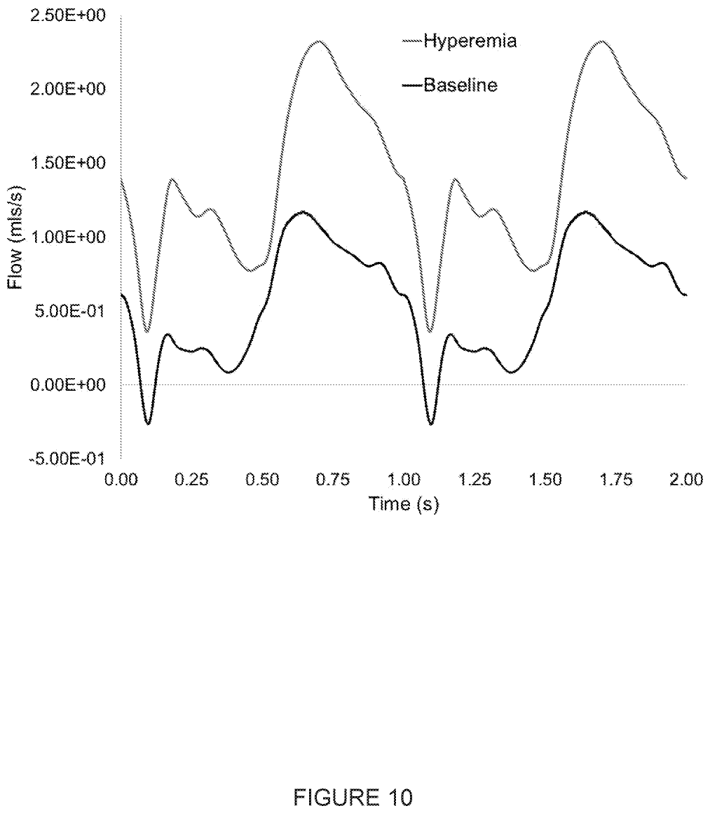

Q_{CFD} was computed in 21 cases under baseline and hyperemic conditions resulting in a total of 42 separate simulations. In each case, Q_{CFD} was used to calculate CMVR and SR. FIG. 7 summarises the pressure, flow (Q_{CFD}), CMVR and SR for each case under baseline and hyperemic conditions and the corresponding CFR derived from the Q_{CFD} results. Between baseline and hyperemic conditions, mean SR was relatively stable (0.18 (0.14) vs 0.22 (0.14) mmHg·s/ml), whereas the CMVR decreased, on average, by 48% (p=0.009). (The following abbreviations are used in FIG. 10: LAD=left anterior descending, RCA=right coronary artery, LCX=left circumflex, dP=delta pressure, Pd/Pa=distal to proximal pressure ratio, FFR=fractional flow reserve, CMVR=coronary microvascular resistance, SR=stenosis resistance, Q_{CFD}−CFR=coronary flow reserve derived from Q_{CFD} results. The following units are used in FIG. 10: pressure=mmHg, flow=ml/min; resistance=mmHg min/ml. As will be appreciated, these units are standard units that are well recognised in the field.)

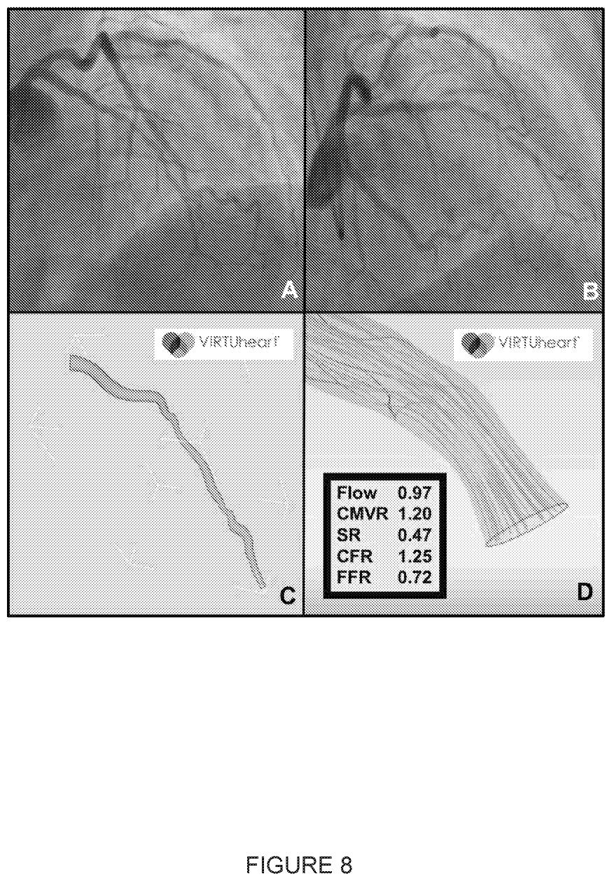

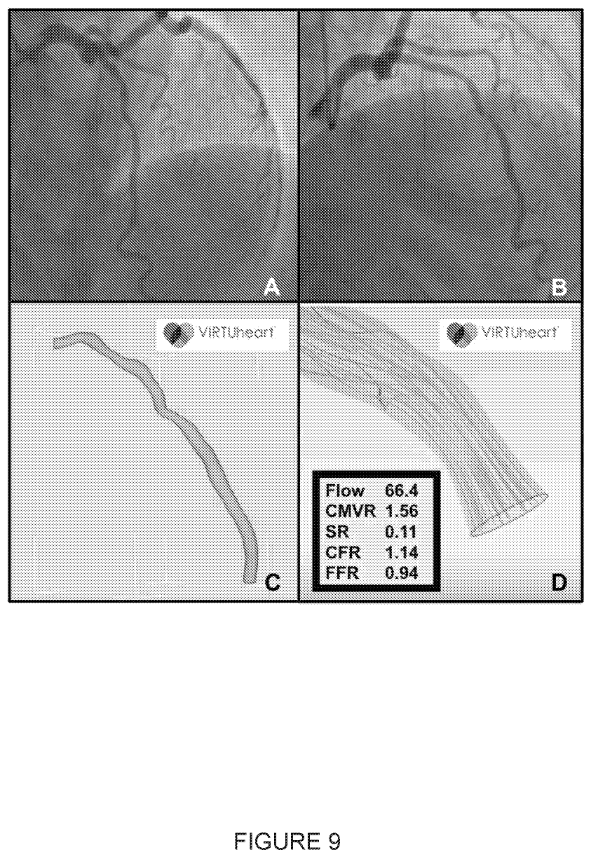

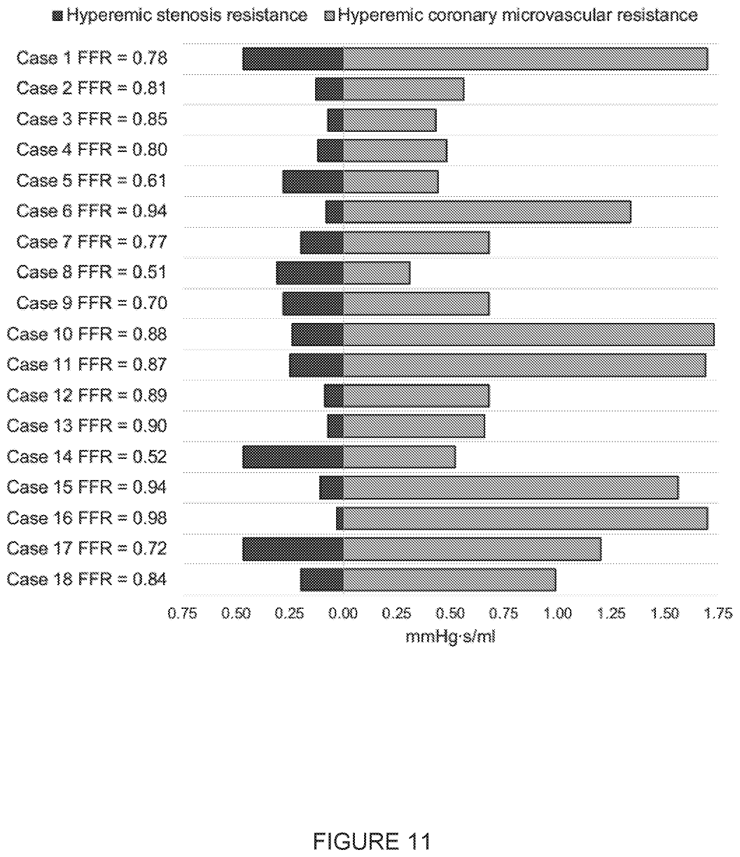

FIGS. 8 and 9 illustrate two representative cases and demonstrate the comprehensive physiological assessment provided by the novel method. Mean CFD processing time was 189 seconds. FIG. 10 demonstrates an output of the Q_{CFD} method for both baseline and hyperemic conditions. FIG. 11 illustrates the total resistance to flow for each case under hyperemic conditions including the relative contribution from the stenosis and microvascular compartments.

### Results: Doppler Flow (Q_{Dop})

In two left anterior descending artery (LAD) cases the Doppler data were of inadequate quality under baseline conditions and CFR was therefore excluded. Doppler- and Q_{CFD}-derived values of CFR were compared on a case-by-case basis in the remaining 19 patient cases. There was wide disagreement between both methods and CFR calculated from Q_{Dop} consistently underestimated Q_{CFD} (mean delta; −0.4 ml/s (1.55), Bland Altman limits of agreement; −3.0 to +3.0). The percentage difference between both methods ranged from −54% to +131% reflecting considerable disagreement between the two methods.

Results: Reversing the Computational Model to Validate Q_{CFD} and Q_{Dop}

When computed Q_{CFD} results were prescribed in the computational model, the simulated pressure gradients were in close agreement with the invasive pressure measurements recorded during cardiac catheterization (bias=−0.29 mmHg; SD, 0.46, p=0.95). When the Q_{Dop} results were prescribed, the resulting pressure gradients consistently underestimated the invasively measured gradient (bias, −8.93 mmHg; SD, 12.46, p<0.01). These results support Q_{CFD} as being an accurate measure of coronary flow and Q_{Dop} being an inaccurate and variable marker of volumetric coronary flow rate.

1. A computer-implemented method of determining data indicating volumetric flow rate within an artery comprising:
receiving data indicating a proximal pressure measurement relative to a direction of blood flow;
receiving data indicating a distal pressure measurement relative to the direction of blood flow;
wherein the proximal pressure measurement and the distal pressure measurement are obtained using at least one pressure transducer;
receiving geometric model data representing the artery, wherein the geometric model data is generated based upon image data indicative of the artery; and
computing a volumetric flow rate within the artery, wherein computing comprises performing a numerical simulation of blood flow through the artery based upon the geometric model data, the data indicating a proximal pressure measurement and the data indicating a distal pressure measurement.

2. The method of claim 1, wherein the numerical simulation is a computational fluid dynamics simulation.

3. The method of claim 1, further comprising:
setting one or more boundary conditions of the numerical simulation based upon the data indicating a proximal pressure measurement and the data indicating a distal pressure measurement.

4. The method of claim 3, wherein setting one or more boundary conditions comprises:
setting an inlet pressure boundary condition based upon the data indicating a proximal pressure measurement; and
setting an outlet pressure boundary condition based upon the data indicating a distal pressure measurement.

5. The method of claim 1, further comprising:
determining one or more physiological parameters associated with the artery based upon the computed volumetric flow rate.

6. The method of claim 5, wherein the one or more physiological parameters comprises one or more of the following: stenosis resistance, distal microvascular resistance, coronary microvascular resistance and coronary flow reserve.

7. The method of claim 1, further comprising:
receiving image data representing the artery;
generating a three dimensional model representing the artery based upon the image data;
discretizing the three dimensional model; and
generating the geometric model data representing the artery based upon the discretized three dimensional model.

8. The method of claim 1, wherein the artery is a coronary artery.

9. The method of claim 8, wherein the image data is based upon a coronary angiogram.

10. The method of claim 1, wherein the at least one pressure transducer comprises a catheter and/or pressure wire.

11. A computer apparatus for determining data indicating volumetric flow rate within an artery comprising:
a memory storing processor readable instructions;
a processor arranged to read and execute instructions stored in said memory; wherein said processor readable instructions comprise instructions arranged to control the computer to carry out a method according to any preceding claim.

12. A computer readable medium carrying computer readable instructions configured to cause a computer to carry out a method according to claim 1.

13. An apparatus for determining data indicating volumetric flow rate within an artery comprising:
a computer readable medium carrying computer readable instructions configured to cause a computer to carry out a method according to claim 1; and
a pressure transducer for obtaining a pressure measurement in an artery.
